(19) United States
(12) Patent Application Publication
(10) Pub. No.: US 2017/0290691 A1
(43) Pub. Date: Oct. 12, 2017

(54) STENT DEPLOYMENT SYSTEM INCLUDING MULTIPLE STENT-ENGAGING ELEMENTS

(71) Applicant: IDev Technologies, Inc., Santa Clara, CA (US)

(72) Inventor: Nianjiong Joan Bei, Palo Alto, CA (US)

(21) Appl. No.: 15/097,013

(22) Filed: Apr. 12, 2016

(51) Int. Cl. A61F 2/966; A61F 2/844

(52) U.S. Cl. CPC A61F 2/966; A61F 2/844; A61F 2002/9665

(57) ABSTRACT

A stent delivery system can include a plurality of stent-engaging elements configured to move relative to each other. Each of the stent-engaging elements can include a radially outwardly projecting portion configured to engage a stent. Each of the stent-engaging elements can be configured to move the stent when distally advanced and not move the stent when proximally retracted.

### BACKGROUND

### Field

The present disclosure generally relates to devices, systems, methods for making, and methods for use in delivering or deploying a stent.

### Description of the Related Art

Body vessels or certain non-body structures such as polymer tubes may have a lumen that is at least partially occluded by a lesion or obstruction. Stent delivery systems can be used to insert a stent to help restore patency to the lumen.

### SUMMARY

Some stent delivery systems can include a pushing mechanism that acts on a proximal end of the stent to drive the stent out of the delivery system. However, this pushing mechanism can be particularly ineffective with long and/or flexible stents. As a result, when a force is applied to the stent in a longitudinal direction, the stent can buckle radially, which can make delivery of the stent difficult. Other stent delivery systems require the sheath to be withdrawn to release the stent from the delivery system. However, with longer stents, retracting the sheath over the required distance can be cumbersome and maintaining the position of the stent at the target location can be difficult. Thus, it can be desirable to engage intermediate portions of the stent and/or to distally drive the stent out of the delivery system in portions less than the entire stent.

Although distally driving portions of the stent can facilitate delivery, this method may require multiple cycles of pushing and pulling in order to fully deploy a stent, which can be onerous on the user and impart a high load on the ratchet, particularly for long stents and/or stents with significant foreshortening upon deployment. Thus, it can be desirable to provide multiple stent-engaging elements that can continually push the stent and/or reduce the total number of movements required by the user. In some instances, with a single movement of a user-actuatable element, the entire length of a particularly long and/or flexible stent can be accurately delivered out of the outer member in sequential portions to a target location.

In general, the stent delivery system can include an elongate outer member and a pusher assembly in a lumen of the elongate outer member. The pusher assembly can include two or more stent-engaging elements, for example a distal (or first) stent-engaging element and a proximal (or second) stent-engaging element. The distal stent-engaging element can include a first radially outwardly projecting portion configured to engage a first portion of a self-expanding stent. The proximal stent-engaging element can include a second radially outwardly projecting portion configured to engage a second portion of the self-expanding stent. The second portion can be proximal to the first portion of the stent-engaging element. The distal stent-engaging element can be configured to move independently from the proximal stent-engaging element.

Various methods of delivering a stent, for example using the above-described system, are described herein to address the problems described above. One method can include advancing the stent delivery system proximate to the treatment site with the self-expanding stent radially between the elongate outer member and the pusher assembly in a radially compressed configuration. The method can also include advancing a first portion of the self-expanding stent out of the elongate outer member by distally driving the first stent-engaging element out of the elongate outer member. After advancing the first portion of the self-expanding stent, a second portion of the self-expanding stent can be advanced out of the elongate outer member by distally driving the second stent-engaging element out of the elongate outer member, while not distally driving the first stent-engaging element.

In another method, a first portion of the self-expanding stent can be advanced out of the elongate outer member by distally driving the first stent-engaging element without distally driving the first stent-engaging element out of the elongate member. After advancing the first portion of the self-expanding stent, the method can include advancing a second portion of the self-expanding stent out of the elongate outer member by distally driving the second stent-engaging element without distally driving the second stent-engaging element out of the elongate member and while not distally driving the first stent-engaging element.

For stent delivery systems including more than two stent-engaging elements, the method can include advancing the plurality of stent-engaging elements until the plurality of stent engaging elements are positioned at a distal portion of the outer lumen. Advancing the plurality of stent-engaging elements can include distally driving each of the at least one intermediate stent-engaging elements and the proximal stent-engaging element, while not distally driving any of the plurality of stent-engaging elements distal to the stent-engaging element being driven. Although in other methods, instead of positioning the plurality of stent-engaging elements at a distal portion of the outer lumen, advancing the plurality of stent-engaging elements can include sequentially advancing each of the stent-engaging elements out of the outer lumen.

Any feature, structure, or step disclosed herein can be replaced with or combined with any other feature, structure, or step disclosed herein, or omitted. Further, for purposes of summarizing the disclosure, certain aspects, advantages, and features of the inventions have been described herein. It is to be understood that not necessarily any or all such advantages are achieved in accordance with any particular embodiment of the inventions disclosed herein. No individual aspects of this disclosure are essential or indispensable.

### BRIEF DESCRIPTION OF THE DRAWINGS

FIGS. 1A-1G schematically illustrate cross-sectional views of a distal potion of an embodiment of a stent delivery system in a method of delivering a stent into a vessel.

FIGS. 2A-2F schematically illustrate cross-sectional views of a distal portion of an embodiment of a delivery system in another method of delivering a stent into a vessel.

FIG. 3A is a perspective view of a distal portion of an embodiment of a pusher assembly.

FIG. 3B is a distal end view of the pusher assembly shown in FIG. 3A.

FIG. 4 schematically illustrates a cross-section of an embodiment of a pusher assembly including a hydraulic mechanism.

FIG. 5 schematically illustrates an embodiment of a stent delivery system including a user-actuatable element.

Various embodiments are depicted in the accompanying drawings for illustrative purposes, and should in no way be interpreted as limiting the scope of the embodiments. Furthermore, various features of different disclosed embodiments can be combined to form additional embodiments, which are part of this disclosure.

### DETAILED DESCRIPTION

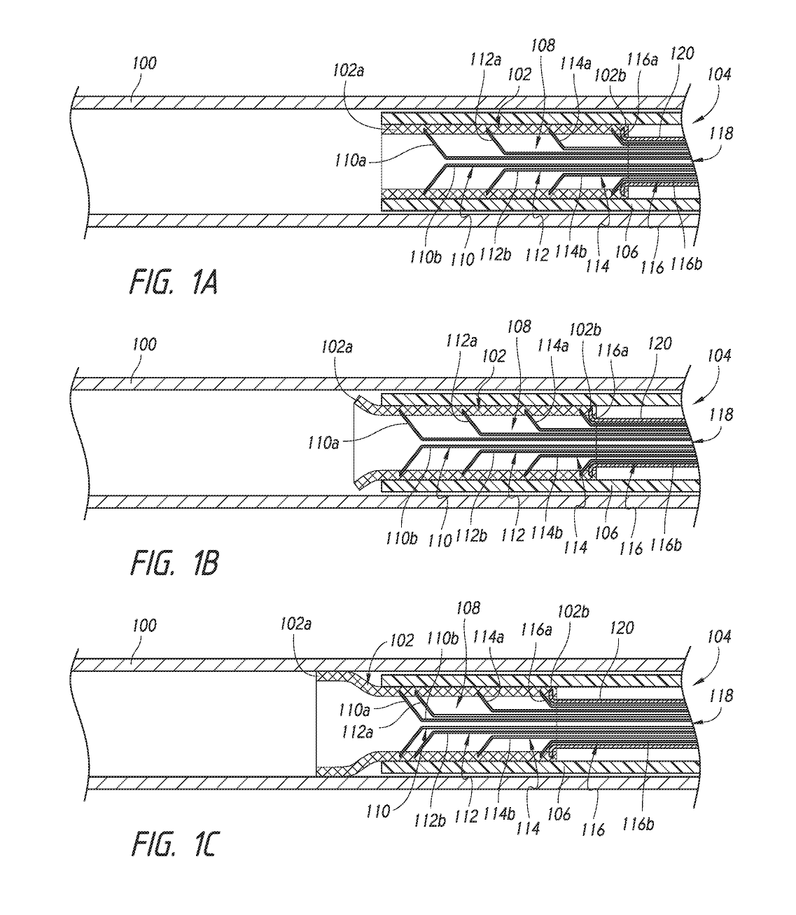

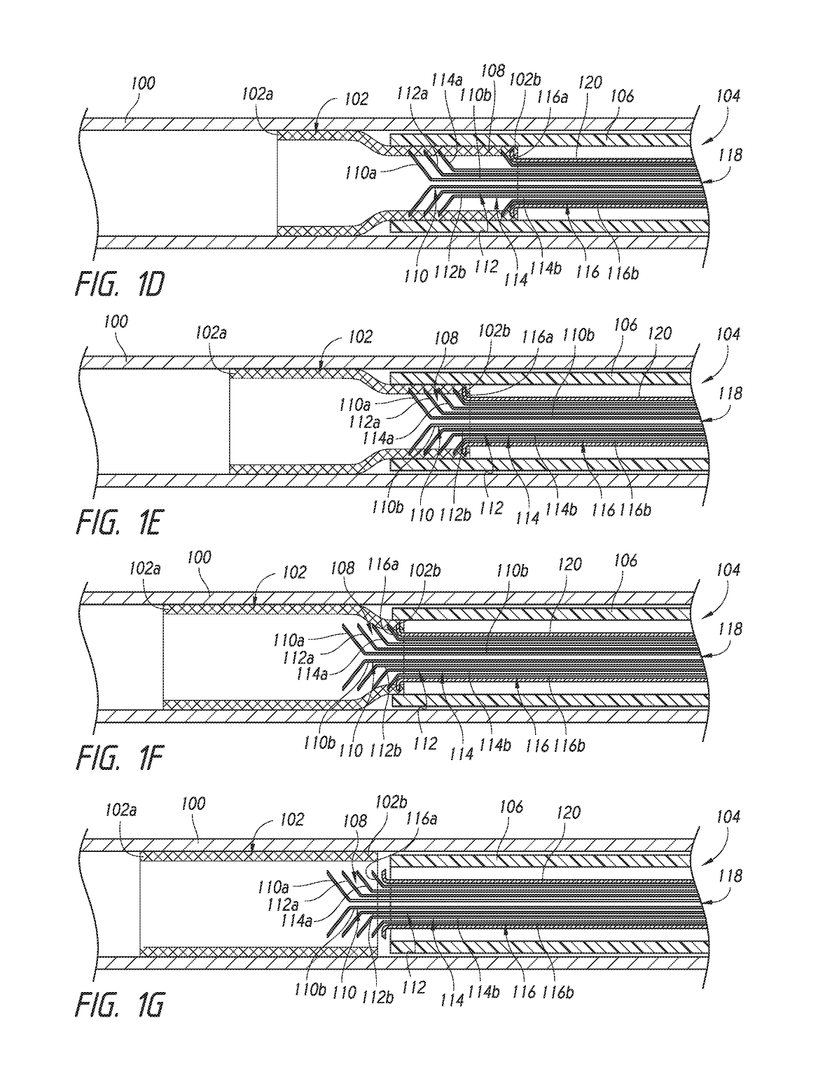

FIGS. 1A-1G schematically illustrate cross-sectional views of a distal potion of an embodiment of a stent delivery system 104 in a method of delivering a stent 102 into a vessel 100 (e.g., body vessel or other non-body structure). The stent delivery system 104 can include an elongate outer member 106 defining an outer member lumen. A pusher assembly 108 can be positioned radially inward of the elongate outer member 106. The pusher assembly 108 can include plurality of stent-engaging elements 110, 112, 114, 116 (e.g., two, three, four, five, six, or more stent-engaging elements).

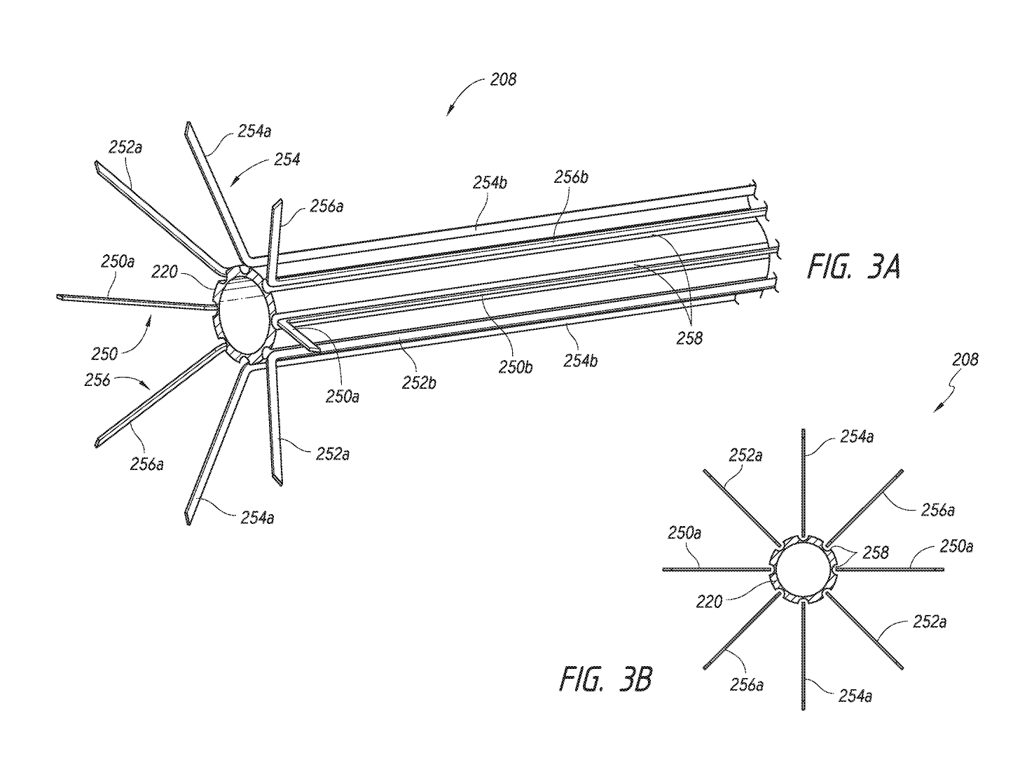

The pusher assembly 108 can include a guidewire channel 118. As shown in FIGS. 1A-1G, at least one of the stent-engaging elements 110, 112, 114, 116 can at least partially define the guidewire channel 118. A separate guidewire channel 220, 320, 420, 520 can be positioned radially inward of the pusher assembly 108 (see FIGS. 3A-5) to at least partially define the guidewire channel 118. A combination of at least one of the stent-engaging elements 110, 112, 114, 116 and a separate guidewire channel 220, 320, 420, 520 can at least partially define the guidewire channel 118.

Before deploying the stent 102, the entire stent 102 can be positioned in the delivery system 104 between the pusher assembly 108 and the outer member 106 (see FIG. 1A). The stent 102 can be positioned in the delivery system 104 when the delivery system 104 is advanced into the vessel 100 (e.g., over a guidewire extending through the guidewire channel 118). Although suitable for deploying several kinds of stents, the systems described herein may be particularly advantageous for self-expanding woven stents such as described in U.S. Pat. No. 7,018,401, filed Feb. 1, 2000, titled “Woven Intravascular Devices and Methods for Making the Same and Apparatus for Delivery of the Same,” and U.S. Patent Publication No. 2008/0290076, filed Oct. 22, 2007, titled “Secured Strand End Devices,” both of which are hereby incorporated by reference in their entirety. For example, the stent 102 can be a self-expanding stent having a distal end 102a and a proximal end 102b. The stent 102 can include a plurality of strands plain woven to form multiple wire intersections. The self-expanding properties may arise from shape-setting of shape memory strands such as nitinol. The delivery systems described herein can be particularly useful because the stent-engaging elements 110, 112, 114, 116 can engage the wire intersections of the stent 102. The delivery systems described herein can also be used to engage struts or residual material of a cut hypotube or rolled sheet.

The pusher assembly 108 can be configured to drive the stent 102 out of the outer member 106. The pusher assembly 108 can include a distal stent-engaging element 110, a proximal stent-engaging element 116, and zero or one or more intermediate stent-engaging elements 112, 114. Although FIGS. 1A-1G illustrate four stent-engaging elements 110, 112, 114, 116, the pusher assembly 108 can include a fewer or greater number of stent-engaging elements depending on, for example, the length of the stent to be deployed, the amount of foreshortening of the stent to be deployed, a tolerable number of actuation engagements, and the like.

Each of the plurality of stent-engaging elements 110, 112, 114, 116 can include a distal portion 110a, 112a, 114a, 116a and a proximal portion 110b, 112b, 114b, 116b. Each of the proximal portions 110b, 112b, 114b, 116b can include a tubular portion. The tubular portion can fully surround the guidewire lumen 118 or only partially surround the guidewire lumen 118, e.g., surround the guidewire lumen by less than or equal to about 180 degrees or less than or equal to about 90 degrees.

Each of the distal portions 110a, 112a, 114a, 116a can extend radially outward from the proximal portions 110b, 112b, 114b, 116b, such that the distal portions 110a, 112a, 114a, 116a can engage the stent 102 (e.g., between wire intersections of a woven stent). As shown in FIGS. 1A-1G, each of the distal portions 110a, 112a, 114a, 116a can include at least one radially projecting prong (e.g., one, two, three, four, five, six, or more prongs). For example, each of the distal portions 110a, 112a, 114a, 116a may include two radially projecting prongs. A first radially projecting prong can be displaced 180 degrees from a second radially projecting prong. For illustration purposes, the distal portions 110a, 112a, 114a, 116a are shown at the same radial position. However, in other configurations, as described below, one or more of the distal portions 110a, 112a, 114a, 116a may be circumferentially offset from the other distal portions (see e.g., FIGS. 3A and 3B).

Each of the stent-engaging elements 110, 112, 114, 116 can move the stent 102 when distally advanced, for example by engaging wire intersections, and not move the stent 102 when proximally retracted, for example by folding or bending inwardly. A material (e.g., shape material alloy) and/or dimension (e.g., thickness of radially projecting prong or other stent-engaging member) may enable the distal portions of the stent-engaging element 110a, 112a, 114a, 116a to fold or bend inward. Additional information regarding other stent-engaging element configurations (e.g., shovel-shaped) and methods of manufacturing can be found in U.S. Pat. No. 8,876,881, filed Oct. 22, 2007, titled “Devices for Stent Advancement,” and U.S. Patent Publication No. 2011/0295354, filed May 27, 2011, titled “Stent Delivery System with Pusher Assembly,” both of which are hereby incorporated by reference in their entirety.

The stent-engaging elements 110, 112, 114, 116 can be arranged along a longitudinal axis of the delivery system 104. As shown in FIG. 1A, the stent-engaging elements 110, 112, 114, 116 can be arranged in a telescoping configuration, such that at least part of a portion of each stent-engaging element 110, 112, 114, 116 is positioned radially inward of the immediately proximal stent-engaging element and radially inwardly of the other proximal stent engaging elements. For example, at least part of a proximal portion 110b of stent-engaging element 110 can be positioned radially inward of the immediately proximal stent-engaging element 112 and radially inwardly of the proximal stent-engaging elements 114, 116. For another example, at least part of a proximal portion 112b of stent-engaging element 112 can be positioned radially inward of the immediately proximal stent-engaging element 114 and radially inwardly of the proximal stent-engaging element 116. For yet another example, at least part of a proximal portion 114b of stent-engaging element 114 can be positioned radially inward of the immediately proximal stent-engaging element 116.

At least a first subset of the stent-engaging elements 110, 112, 114, 116 can be configured to move relative a second subset of the stent-engaging elements 110, 112, 114, 116. For example, as shown in FIG. 1B, the first subset of stent-engaging elements can include the distal stent-engaging element 110, and the second subset of stent-engaging elements can include the intermediate and proximal stent-engaging elements 112, 114, 116. The second subset of stent-engaging elements can be driven distally, while not driving the first subset of stent-engaging elements. Other combinations of first and second subsets of stent-engaging elements can be seen in FIGS. 1C-1E.

As shown in FIGS. 1B-1E, each of the stent-engaging elements 110, 112, 114, 116 can be driven distally without being driven out of the elongate outer member 106. The distance at which the stent-engaging elements 110, 112, 114, 116 attributed to a length of the stent-engaging elements 110, 112, 114, 116 and/or a stop feature (e.g., on the stent-engaging element or in a handle of the delivery system 104) that prevents the stent-engaging elements 110, 112, 114, 116 from being driven out of the elongate outer member 106. This stop feature can inhibit or prevent the proximal end 102b of the stent 102 from being driven out of the elongate outer member, such that the stent 102 may be retracted, if necessary, as described below. As described in further detail below, the handle can include one or more stoppers that inhibit movement of a user-actuatable element and thus limit movement of the stent-engaging elements 110, 112, 114, 116.

In use, after advancing the delivery system 102 proximate to the treatment site (see FIG. 1A), the stent-engaging elements 110, 112, 114, 116 can be distally advanced until the stent-engaging elements 110, 112, 114, 116 reach their respective stop positions (FIGS. 1B-1E). As the stent-engaging elements 110, 112, 114, 116 distally advance, the telescope configuration of the pusher assembly 108 is collapsed. The distance between the distal portions 110b, 112b, 114b, 116b of stent-engaging elements 110, 112, 114, 116 can decrease as the stent-engaging elements 110, 112, 114, 116 are distally advanced. After each of the stent-engaging elements 110, 112, 114, 116 has been distally advanced to its respective stop position, the stent-engaging elements 110, 112, 114, 116 can be simultaneously distally advanced to distally advance a proximal end 102b of the stent 102 out of the elongate outer member 106 (see FIGS. 1F and 1G), or, if enough of the stent 102 remains in the outer member 106, at least some of the stent-engaging elements 110, 112, 114, 116 can be proximally retracted (e.g., to the position shown in FIG. 1A) and again distally advanced, one or more times, before being distally advanced to distally advance the proximal end 102b of the stent 102 out of the elongate outer member 106.

In the method shown in FIGS. 1A-1G, a first portion of the self-expanding stent 102 can be advanced out of the elongate outer member 106 by distally driving the stent-engaging elements 110, 112, 114, 116 until the distal stent-engaging element 110 reaches a first stop position (see FIG. 1B). After advancing the first portion of the self-expanding stent 102, a second portion of the self-expanding stent 102 can be advanced out of the elongated member 106 by distally driving the intermediate and proximal stent-engaging elements 112, 114, 116, while not distally driving the distal stent-engaging element 110 (see FIG. 1C). The intermediate and proximal stent-engaging elements 112, 114, 116 can be driven distally until the intermediate stent-engaging element 112 reaches a second stop position. Next, a third portion of the self-expanding stent 102 can be advanced out of the elongate outer member 106 by distally driving the intermediate stent-engaging element 114 and the proximal stent-engaging element 116, while not distally driving the distal stent-engaging element 110 and the intermediate stent-engaging element 112 (see FIG. 1D). The intermediate stent-engaging element 114 and the proximal stent-engaging element 116 can be driven distally until the intermediate stent-engaging element 114 reaches a third stop position. Thereafter, a fourth portion of the self-expanding stent 102 can be advanced out of the elongate outer member 106 by distally driving a proximal stent-engaging element 116, while not distally driving the distal and intermediate stent-engaging elements 110, 112, 114 distal to the proximal stent-engaging element 116 (FIG. 1E). The proximal stent-engaging element 116 can be driven distally until the proximal stent-engaging element 116 reaches a fourth stop position. At least the proximal stent-engaging element 116 can be configured such that a distal portion 116a of the stent-engaging element 116 remains within the outer member 106 when the proximal stent-engaging element 116 reaches the fourth stop position, which may allow the stent 102 to be retracted back into the outer member 106 by a stent retention element 120 (described in more detail below) and possibly repositioned, if desired. After the proximal stent-engaging element 116 has been driven distally to the fourth stop position, the stent-engaging elements 110, 112, 114, 116 can be driven (e.g., simultaneously) to deliver the proximal end 102b of the stent 102 out of the elongate outer member 106 (see FIGS. 1F and 1G).

As each of the stent-engaging elements 110, 112, 114, 116 is being driven distally, the portion of the stent 102 being driven is advanced over the stent-engaging elements distal to the stent-engaging element being driven. For example, as the intermediate and proximal stent-engaging elements 112, 114, 116 are driven distally, the second portion of the self-expanding stent 102 can be advanced over the distal stent-engaging element 110. The distal portion 110a of the distal stent-engaging element 110 can bend or flex inward as the second portion of the self-expanding stent 102 is advanced over the distal portion 110a.

For longer stents, prior to driving the proximal end 102b of the stent 102 out of the elongate outer member 106, a user can retract one or more of the stent-engaging elements 110, 112, 114, 116, so the stent-engaging elements 110, 112, 114, 116 can be re-advanced to distally drive additional portions of the stent 102 out of the elongate outer member 106. When proximally retracted, the distal portions 110a, 112a, 114a, 116a can flex or bend inwardly and slide along an inner surface of the stent 102, such that the stent-engaging elements 110, 112, 114, 116 can be retracted without moving (e.g., distally advancing and/or proximally retracting) the stent 102.

The delivery system 104 can include a stent retention element 120 that allows the operator to retract the stent 102 into the outer member 106, provided that the stent 102 has not been advanced completely out of the outer member 106. If the stent retention element 120 moves distally out of the outer member 106, the proximal end of the stent 102b will be deployed and cannot be retracted.

After the stent-engaging elements 110, 112, 114, 116 have been deployed out of the outer member 106 and prior to deploying the stent retention element 120 out of the outer member 106 (see FIG. 1F), the stent retention element 120 can be proximally retracted to retract the stent 102 back into the outer member 106. The stent-engaging elements 110, 112, 114, 116 do not inhibit or prevent the proximal retraction of the stent 102 because, being out of the outer member 106, they are distal enough to not interact with the stent 102 as the stent 102 is being withdrawn. After the stent 102 has been withdrawn back into the outer member 106, the stent-engaging elements 110, 112, 114, 116 can be proximally retracted into the outer member 106 and through the stent 102. As the stent-engaging elements 110, 112, 114, 116 are proximally retracted through the stent 102, the distal portions 110a, 112a, 114a, 116a of the stent-engaging elements 110, 112, 114, 116 can bend or flex inwardly and slide along the inner surface of the stent 102 so as to not disturb a position of the stent 102 in the outer member 106. After the stent 102 and stent-engaging elements 110, 112, 114, 116 have been withdrawn back into the outer member 106 and through the stent 102, the stent 102 can be re-deployed, for example in the manner described above. In other configurations, the stent retention element 120 and the stent-engaging elements 110, 112, 114, 116 can be simultaneously withdrawn. In some configurations in which the stent-engaging elements 110, 112, 114, 116 comprise shape memory material, warm or cold fluid (e.g., saline) can be used to cause the distal portions 110a, 112a, 114a, 116a of the stent-engaging elements 110, 112, 114, 116 to change shape radially inwardly such that the distal portions 110a, 112a, 114a, 116a do not inhibit or prevent the proximal retraction of the stent 102. Additional information regarding stent retention elements can be found in U.S. Pat. No. 8,876,881, filed May 27, 2011, titled “Stent Delivery System with Pusher Assembly,” which is hereby incorporated by reference in its entirety.

Although FIGS. 1A-1G are drawn with a stent retention element 120, the delivery system 104 may lack or not include a stent retention element. Instead, once the proximal stent-engaging element 116 moves out of the outer member 106, the proximal end 102b of the stent 102 will be released from the outer member 106.

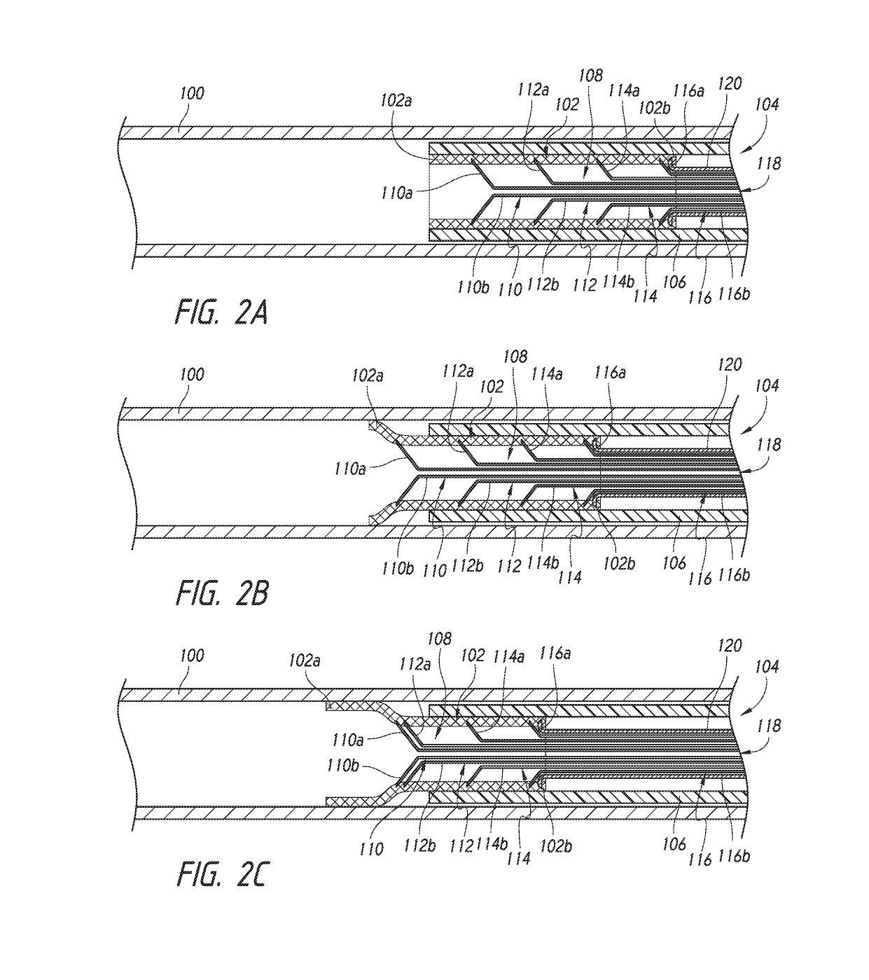

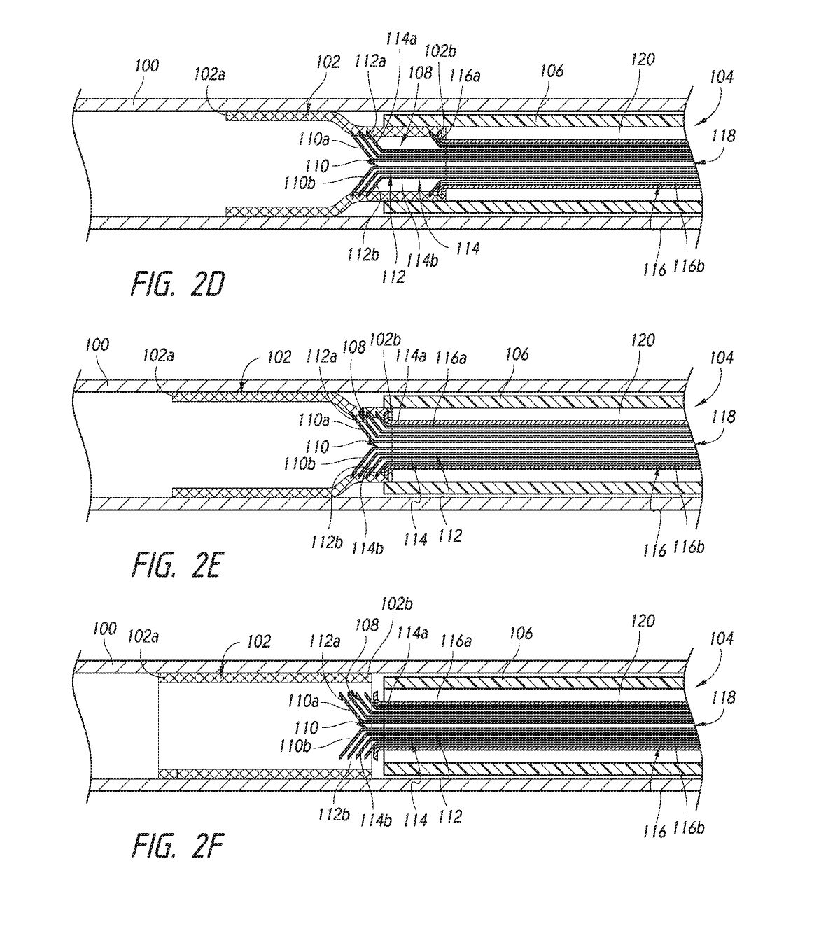

FIGS. 2A-2F schematically illustrate cross-sectional views of a distal potion of an embodiment of a stent delivery system 104 in another method of delivering a stent 102 into a vessel 100 (e.g., body vessel or other non-body structure). The stent delivery system 104 can include any of the features described above with respect to FIGS. 1A-1G, including a plurality of stent-engaging elements 110, 112, 114, 116 (e.g., two, three, four, five, six, or more stent-engaging elements). For illustration purposes, the method shown in FIGS. 2A-2F shows four stent-engaging elements 110, 112, 114, 116, but a fewer or greater number of stent-engaging elements may be used.

Similar to the previous method, at least a first subset of the stent-engaging elements 110, 112, 114, 116 can be configured to move relative a second subset of the stent-engaging elements 110, 112, 114, 116. For example, as shown in FIG. 2C, the first subset of stent-engaging elements can include the distal stent-engaging element 110, and the second subset of stent-engaging elements can include the intermediate and proximal stent-engaging elements 112, 114, 116. The second subset of stent-engaging elements can be driven distally, while not driving the first subset of stent-engaging elements. Other combinations of first and second subsets of stent-engaging elements can be seen in FIGS. 2D-2E. However, in contrast to the previous method, each stent-engaging element 110, 112, 114, 116 can be driven distally out of the elongate outer member 106 in a sequential manner.

In use, after advancing the delivery system 102 proximate to the treatment site (see FIG. 2A), the stent-engaging elements 110, 112, 114, 116 can be distally advanced until the stent-engaging elements 110, 112, 114, 116 reach their respective stop positions outside the outer member 106 (FIGS. 2B-2E). As the stent-engaging elements 110, 112, 114, 116 distally advance, the telescope configuration of the pusher assembly 108 is collapsed. The distance between the distal portions 110b, 112b, 114b, 116b of stent-engaging elements 110, 112, 114, 116 can change as the stent-engaging elements 110, 112, 114, 116 are distally advanced.

In the method shown in FIGS. 2A-2F, a first portion of the self-expanding stent 102 can be advanced out of the elongate outer member 106 by distally driving the stent-engaging elements 110, 112, 114, 116 until the distal stent-engaging element 110 reaches a first stop position out of the outer member 106, e.g., a distal portion 110a of stent-engaging element 110 is distal to a distal end of the outer member 106 (see FIG. 2B). During this step, the spacing between the distal portions 110a, 112a, 114a, 116a of the stent-engaging elements 110, 112, 114, 116 remains unchanged.

After advancing the first portion of the self-expanding stent 102, a second portion of the self-expanding stent 102 can be advanced out of the elongated member 106 by distally driving the intermediate and proximal stent-engaging elements 112, 114, 116, while not distally driving the distal stent-engaging element 110 (see FIG. 2C). The intermediate and proximal stent-engaging elements 112, 114, 116 can be driven distally until the intermediate stent-engaging element 112 reaches a second stop position out of the outer member 106, e.g., a distal portion 112a of stent-engaging element 112 is distal to a distal end of the outer member 106 (see FIG. 2C). As the intermediate and proximal stent-engaging elements 112, 114, 116 are driven distally, the spacing between their respective distal portions 112a, 114a, 116a can remain the same, while the spacing between the distal portion 110a of distal stent-engaging element 110 and the distal portion 112a of the intermediate stent-engaging element 112 decreases.

Next, a third portion of the self-expanding stent 102 can be advanced out of the elongate outer member 106 by distally driving the intermediate stent-engaging element 114 and the proximal stent-engaging element 116, while not distally driving the distal stent-engaging element 110 and the intermediate stent-engaging element 112 (see FIG. 2D). The intermediate stent-engaging element 114 and the proximal stent-engaging element 116 can be driven distally until the intermediate stent-engaging element 114 reaches a third stop position out of the outer member 106, e.g., a distal portion 114a of stent-engaging element 114 is distal to a distal end of the outer member 106 (see FIG. 2D). As the intermediate and proximal stent-engaging elements 114, 116 are driven distally, the spacing between their respective distal portions 114a, 116a can remain the same, while the spacing between the distal portion 112a of intermediate stent-engaging element 112 and the distal portion 114a of the intermediate stent-engaging element 114 decreases.

Thereafter, a fourth portion of the self-expanding stent 102 can be advanced out of the elongate outer member 106 by distally driving a proximal stent-engaging element 116, while not distally driving the distal and intermediate stent-engaging elements 110, 112, 114 distal to the proximal stent-engaging element 116 (FIG. 2E). The proximal stent-engaging element 116 can be driven distally until the proximal stent-engaging element 116 reaches a fourth stop position out of the outer member 106, e.g., a distal portion 116a of stent-engaging element 116 is distal to a distal end of the outer member 106 (see FIG. 2E). During this step, the spacing between the distal portion 114a of intermediate stent-engaging element 114 and the distal portion 116a of proximal stent-engaging element 116 decreases. The fourth portion of the stent or the portion advanced by the proximal stent-engaging element 116 can include a proximal end of the stent 102b, such that distally advancing the proximal stent-engaging element 116 releases the entire stent 102 from the outer member 106. Although in other configurations, the stent delivery system may include a stent retention element 120 that permits the stent 102 to be retracted back into the outer member 106 for repositioning. If the stent 102 is properly positioned in the vessel, the stent retention element 120 may be distally advanced out of the outer member 106 to release the entire stent 102 from the outer member 106 (see FIG. 2F). The stent engaging elements 110, 112, 114, 116 may also be withdrawn to redeploy the stent as described above.

As each of the stent-engaging elements 110, 112, 114, 116 is being driven distally, the portion of the stent 102 being driven is advanced over the stent-engaging elements distal to the stent-engaging element being driven. For example, as the intermediate and proximal stent-engaging elements 112, 114, 116 are driven distally, the second portion of the self-expanding stent 102 can be advanced over the distal stent-engaging element 110.

As described above, the proximal portions of the stent-engaging elements 110b, 112b, 114b, 116b can be telescoping and tubular. In other configurations, the stent-engaging elements 250, 252, 254, 256 can include one or more longitudinal struts (e.g., one, two, three, or more longitudinal struts), for example, each stent-engaging element 250, 252, 254, 256 can include two longitudinal struts 250b, 252b, 254b, 256b. FIG. 3A is a perspective view of a distal portion of an embodiment of a pusher assembly 208 including the longitudinal struts 250b, 252b, 254b, 256b. FIG. 2B is a distal end view of the pusher assembly 208 shown in FIG. 3A. At least one radially outwardly projecting prong 250a, 252a, 254a, 256a can extend distally from each of the longitudinal struts 250b, 252b, 254b, 256b, respectively.

All of the longitudinal struts 250b, 252b, 254b, 256b can be circumferentially displaced from each other around (e.g., surrounding) the guidewire channel 218, such that each distal portion 250a, 252a, 254a, 256a is circumferentially offset from the other distal portions (e.g., evenly spaced around a circumference of the guidewire channel 220 or from each other). In other configurations, a first subset of the radially projecting prongs (e.g., one or more) may be positioned at a first radial position, while a second subset of radially projecting prongs (e.g., one or more) is circumferentially offset from the first position.

When the stent-engaging elements 250, 252, 254, 256 each have multiple longitudinal struts 250b, 252b, 254b, 256b, the longitudinal struts 250b, 252b, 254b, 256b can be evenly spaced apart around a circumference of the pusher assembly 208, such that each portion of the stent is circumferentially pushed out at a same rate. For example, as shown in FIG. 3A, the longitudinal struts 250b, 252b, 254b, 256b of each stent-engaging element 250, 252, 254, 256 can be spaced apart by about 180 degrees, so laterally opposing sides of the stent are pushed out at a same rate. For another example, the longitudinal struts 250b, 252b, 254b, 256b can be positioned such that longitudinal struts 250b, 252b, 254b, 256b of the same stent-engaging element 250, 252, 254, 256 can be adjacent to each other. For yet another example, the longitudinal struts 250b, 252b, 254b, 256b of each of the stent-engaging elements 250, 252, 254, 256 can be spaced apart by about 30 degrees, about 45 degrees, about 60 degrees, about 75 degrees, or otherwise.

In some configurations, each of the longitudinal struts 250b, 252b, 254b, 256b can be tapered in a distal direction to provide more column strength to move each of the stent-engaging elements 250, 252, 254, 256. A width, arc length, diameter, or thickness at a proximal portion of each of the longitudinal struts 250b, 252b, 254b, 256b can be greater than the width, arc length, diameter, or thickness at a distal portion of each of the longitudinal struts 250b, 252b, 254b, 256b. An outer diameter surrounding a proximal portion of the pusher assembly 208 can be greater than an outer diameter surrounding a distal portion of pusher assembly 208, for example because of the tapering of the longitudinal struts 250b, 252b, 254b, 256b.

The guidewire channel 220 can include a number of passageways 258 (e.g., grooves, channels, or otherwise) along which the longitudinal struts 250b, 252b, 254b, 256b can move. The longitudinal struts 250b, 252b, 254b, 256b can have a same length, such that radially projecting prongs 250a, 252a, 254a, 256a are circumferentially aligned prior to delivering the stent (see FIG. 3A). In other configurations, the longitudinal struts 250b, 252b, 254b, 256b can have different lengths, such that the radially projecting prongs 250a, 252a, 254a, 256a are staggered along a longitudinal axis of the pusher assembly 208.

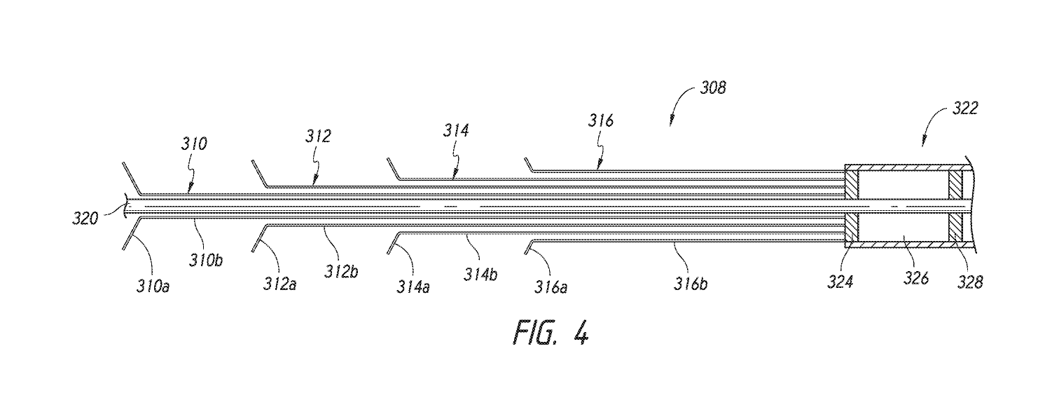

In any of the delivery systems described above, each of the stent-engaging elements can be mechanically coupled to an actuation mechanism in a handle portion of the delivery system. The stent-engaging elements can be directly or indirectly coupled to the actuation mechanism. FIG. 4 schematically illustrates a cross-section of a pusher assembly 308 including a hydraulic mechanism 322. Each of the stent-engaging elements 310, 312, 314, 316 can be bonded to a distal seal 324 of the hydraulic mechanism 322. Moving the proximal stop 328 can increase hydraulic pressure in the hydraulic chamber 326 and cause the distal seal 324 to move one or more of the stent-engaging elements 310, 312, 314, 316 distally. A guidewire channel 320 can extend through the pusher assembly 308, including the hydraulic mechanism 322. The hydraulic mechanism 322 can be actuated as is known for actuation of hydraulic mechanisms in the art. Upon actuation of the hydraulic mechanism 322, the stent-engaging elements 310, 312, 314, 316 can be distally advanced, for example as described above with respect to FIGS. 1A-1F. For illustration purposes, the distal portions 310a, 312a, 314a, 316a are shown at the same radial position. However, in other configurations, as described above, one or more of the distal portions 310a, 312a, 314a, 316a may be circumferentially offset from the other distal portions (see e.g., FIGS. 3A and 3B).

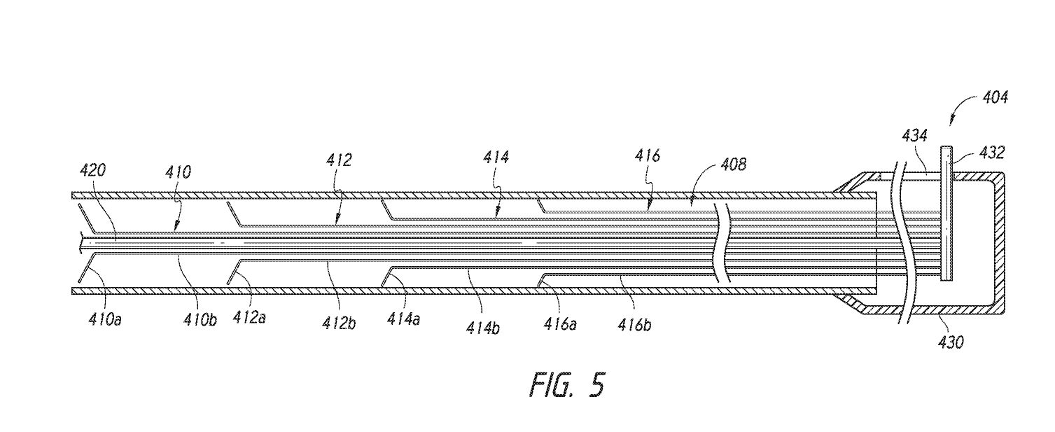

As another example, FIG. 5 schematically illustrates a stent-delivery system 404 including a user-actuatable element 432. Each of the stent-engaging elements 410, 412, 414, 416 can be secured to the user-actuatable element 432 in a handle portion 430 (see FIG. 5). Moving the user-actuatable element 432 along a length of the passageway 434 can move one or more of the stent-engaging elements 410, 412, 414, 416.

The handle portion 430 can include a number of stoppers (not shown), e.g., one, two, etc., that can limit movement of the user-actuatable element 432. A first stopper can limit (e.g., inhibit, prevent) movement of the user-actuatable element 432 (e.g., during shipment, prior to deploying the stent, etc.). After the first stopper has been released, the user-actuatable element 432 can traverse at least a partial length of the passageway 434. The user-actuatable element 432 can move distally or proximally along the partial length of the slot 434 to advance the stent-engaging elements 410, 412, 414, 416.

A second stopper can inhibit or prevent movement of the user-actuatable element 432 along the full length of the passageway 434. If the delivery system 404 includes a stent retention element, the second stopper can be useful for inhibiting or preventing unintentional deployment of the proximal end of the stent. When the second stopper is removed or released, the user-actuatable element 432 can traverse the full length of the passageway 434. When the user-actuatable element 432 traverses the full length of the passageway 434, the stent-engaging elements 410, 412, 414, 416 can push the proximal end of the stent out of the outer member 406 (see FIG. 1F), thereby releasing the stent from the stent retention element (if present) and fully deploying the stent.

For illustration purposes, the distal portions 410a, 412a, 414a, 416a are shown at the same radial position. However, in other configurations, as described above, one or more of the distal portions 410a, 412a, 414a, 416a may be circumferentially offset from the other distal portions (see e.g., FIGS. 3A and 3B).

### Terminology

Although certain delivery systems have been described herein in connection with a plain woven, self-expanding stent, the delivery systems described herein can be used to deliver self-expanding stents or balloon expandable stents having any configuration (e.g., woven, laser cut, or non-plain woven) and constructed from any material (e.g., metal or polymer). For example, in some embodiments, the stent can include a plurality of biodegradable filaments (e.g., made from polyglycolic acid, poly-L-lactic acid, polyorthester, polyanhydride, polyiminocarbonate, or calcium phosphate) woven together to form a self-expanding stent. In some embodiments, the stent can be coated with a drug or the strands can incorporate the drug.

As used herein, the relative terms “proximal” and “distal” shall be defined from the perspective of the delivery system. Thus, proximal refers to the direction of the handle portion of the delivery system and distal refers to the direction of the distal tip.

Note that the terms “first” and “second” stent-engaging elements can be used interchangeably and to refer to any of the stent-engaging elements in the delivery system. In one embodiment, a distal portion of the first stent-engaging element can be distal to the second stent-engaging element. Additional stent-engaging elements may be distal, intermediate, or proximal of the first and second stent-engaging elements.

The terms “first” and “second” portion of the stent can be used interchangeably and to refer to any portion of the stent. In one embodiment, the first portion of the stent can be distal to the second portion of the stent. Additional stent portions may be distal, intermediate, or proximal of the first and second stent portions.

Conditional language used herein, such as, among others, “can,” “might,” “may,” “e.g.,” and the like, unless specifically stated otherwise, or otherwise understood within the context as used, is generally intended to convey that some embodiments include, while other embodiments do not include, certain features, elements, and/or states. Thus, such conditional language is not generally intended to imply that features, elements, blocks, and/or states are in any way required for one or more embodiments or that one or more embodiments necessarily include logic for deciding, with or without author input or prompting, whether these features, elements and/or states are included or are to be performed in any particular embodiment.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Although certain embodiments and examples have been described herein, it will be understood by those skilled in the art that many aspects of the delivery systems shown and described in the present disclosure may be differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Moreover, while illustrative embodiments have been described herein, the scope of any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to the examples described in the present specification or during the prosecution of the application, which examples are to be construed as non-exclusive. Further, the actions of the disclosed processes and methods may be modified in any manner, including by reordering actions and/or inserting additional actions and/or deleting actions. It is intended, therefore, that the specification and examples be considered as illustrative only, with a true scope and spirit being indicated by the claims and their full scope of equivalents.

Any methods disclosed herein need not be performed in the order recited. The methods disclosed herein include certain actions taken by a practitioner; however, they can also include any third-party instruction of those actions, either expressly or by implication. For example, actions such as “retracting the self-expanding stent” include “instructing retraction of the self-expanding stent.”

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±1%, ±5%, ±10%, ±15%, etc.). For example, “about 180 degrees” includes “180 inches.” Phrases preceded by a term such as “substantially” include the recited phrase and should be interpreted based on the circumstances (e.g., as much as reasonably possible under the circumstances).

### Example Embodiments

The following example embodiments identify some possible permutations of combinations of features disclosed herein, although other permutations of combinations of features are also possible.
1. A method of delivering a self-expanding stent at a treatment site using a stent delivery system, the method comprising:
   advancing the stent delivery system proximate to the treatment site, the stent delivery system comprising:
      an elongate outer member defining an outer member lumen;
      a pusher assembly in the outer member lumen, the pusher assembly comprising:
         a first stent-engaging element; and
         a second stent-engaging element, a distal end of the first stent-engaging element positioned distal to the second stent-engaging element, the first stent-engaging element configured to move independently from the second stent-engaging element; and
      the self-expanding stent radially between the elongate outer member and the pusher assembly in a radially compressed configuration;
   advancing a first portion of the self-expanding stent out of the elongate outer member by distally driving the first stent-engaging element; and
   after advancing the first portion of the self-expanding stent, advancing a second portion of the self-expanding stent out of the elongate outer member by distally driving the second stent-engaging element, while not distally driving the first stent-engaging element.
2. The method of Embodiment 1, wherein advancing the first portion of the self-expanding stent comprises distally driving the first stent-engaging element without driving the first stent-engaging element out the elongate outer member.
3. The method of Embodiment 1 or 2, further comprising advancing a third portion of the self-expanding stent out of the elongate outer member by driving a third stent-engaging element.
4. The method of Embodiment 3, wherein advancing the third portion of the self-expanding stent comprises, after advancing the second portion of the self-expanding stent, distally driving the third stent-engaging element, while not distally driving the second stent-engaging element.
5. The method of Embodiment 3, wherein advancing the third portion of the self-expanding stent comprises simultaneously distally driving the second stent-engaging element and the third stent-engaging element.
6. The method of any one of Embodiments 3 to 5, wherein the third portion of the self-expanding stent comprises a proximal end of the self-expanding stent.
7. The method of any one of Embodiments 3 to 6, wherein advancing the third portion of the self-expanding stent comprises driving the third stent-engaging element out of the elongate outer member.
8. The method of any one of Embodiments 1 to 7, wherein the self-expanding stent is woven to form wire intersections, and wherein engaging the self-expanding stent comprises engaging wire intersections of the stent with the first stent-engaging element.
9. The method of any one of Embodiments 1 to 8, wherein advancing the second portion of the stent comprises advancing the second portion of the stent over the first stent-engaging element.
10. The method of any one of Embodiments 1 to 9, further comprising retracting the self-expanding stent.
11. The method of Embodiment 10, wherein retracting the self-expanding stent comprises retracting the first stent-engaging element and the second stent-engaging element.
12. The method of any one of Embodiments 1 to 11, wherein distally driving the first stent-engaging element comprises hydraulically driving the first-stent-engaging element and the second stent-engaging element.
13. The method of any one of Embodiments 1 to 12, further comprising actuating a user-actuatable element configured to drive the first stent-engaging element and the second stent-engaging element.
14. A method of delivering a self-expanding stent at a treatment site using a stent delivery system, the method comprising:
   advancing the stent delivery system proximate to the treatment site, the stent delivery system comprising:
      an elongate outer member defining an outer member lumen;
      a pusher assembly in the outer member lumen, the pusher assembly comprising a plurality of stent-engaging elements, the plurality of stent-engaging elements comprising a distal stent-engaging element, at least one intermediate stent-engaging element, and a proximal stent-engaging element, the plurality of stent-engaging elements being positioned coaxially relative to each other, each of the plurality of stent-engaging elements being configured to move independently relative to the other stent-engaging elements; and
      the self-expanding stent radially between the elongate outer member and the pusher assembly in a radially compressed configuration;
   advancing the plurality of stent-engaging elements until the plurality of stent engaging elements are positioned at a distal portion of the outer lumen, wherein advancing the plurality of stent-engaging elements comprises:
      distally driving each of the at least one intermediate stent-engaging elements and the proximal stent-engaging element, while not distally driving any of the plurality of stent-engaging elements distal to the stent-engaging element being driven.
15. The method of Embodiment 14, wherein advancing the plurality of stent-engaging elements comprises simultaneously distally driving the plurality of stent-engaging elements.
16. The method of Embodiment 14 or 15, wherein advancing the plurality of stent-engaging elements comprises distally driving the plurality of stent-engaging elements toward a distal portion of the outer lumen without driving the plurality of stent-engaging elements out the elongate outer member.
17. The method of Embodiment 16, further comprising, after distally driving the plurality of stent-engaging elements toward the distal portion of the outer lumen, simultaneously driving the plurality of stent-engaging elements out of the elongate outer member.
18. The method of any one of Embodiments 14 to 17, wherein advancing the plurality of stent-engaging elements comprises engaging the self-expanding stent with each of the stent-engaging elements.
19. The method of Embodiment 18, wherein the self-expanding stent is woven to form wire intersections, and wherein engaging the self-expanding stent comprises engaging wire intersections of the stent with each of the stent-engaging element.
20. The method of any one of Embodiments 14 to 19, wherein distally driving each of the at least one intermediate stent-engaging elements and the proximal stent-engaging elements comprises driving a portion of the self-expanding stent over the distal stent-engaging element.
21. The method of any one of Embodiments 14 to 20 further comprising retracting the self-expanding stent.
22. The method of Embodiment 21, wherein retracting the self-expanding stent comprises retracting the plurality of stent-engaging elements.
23. The method of any one of Embodiments 14 to 22, wherein advancing the plurality of stent-engaging elements comprises hydraulically driving the plurality of stent-engaging elements.
24. The method of any one of Embodiments 14 to 23, wherein advancing the plurality of stent-engaging elements comprises actuating a user-actuatable element configured to distally drive the plurality of stent-engaging elements.
25. A stent delivery system comprising:
   an elongate outer member defining an outer member lumen;
   a pusher assembly in the outer member lumen, the pusher assembly comprising:
      a distal stent-engaging element comprising a first radially outwardly projecting portion configured to engage a first portion of a self-expanding stent; and
      a proximal stent-engaging element comprising a second radially outwardly projecting portion configured to engage a second portion of the self-expanding stent, the second portion being proximal to the first portion of the stent-engaging element,
      wherein the distal stent-engaging element is configured to move independently from the proximal stent-engaging element.
26. The stent delivery system of Embodiment 25, wherein the pusher assembly further comprises an intermediate stent-engaging element, a distal end of the intermediate stent-engaging element positioned longitudinally between the distal and the proximal stent-engaging elements, the intermediate stent-engaging element configured to move independently from the distal and the proximal stent-engaging element.
27. The stent delivery system of Embodiment 25 or 26, wherein the distal stent-engaging element is configured to move simultaneously with the proximal stent-engaging element.
28. The stent delivery system of any one of Embodiments 25 to 27, wherein the distal stent-engaging element is coaxial with the proximal stent-engaging element.
29. The stent delivery system of any one of Embodiments 25 to 28, wherein a proximal portion of the distal stent-engaging element is positioned radially inward of the proximal stent-engaging element.
30. The stent delivery system of any one of Embodiments 25 to 29, wherein a proximal portion of distal stent-engaging element is tubular, and wherein a proximal portion of the proximal stent-engaging element is tubular.
31. The stent delivery system of any one of Embodiments 25 to 30, wherein the distal stent-engaging element is longer than the proximal stent-engaging element.
32. The stent delivery system of any one of Embodiments 25 to 28, 30, or 31, wherein a proximal portion of the distal stent-engaging element and a proximal portion of the proximal stent-engaging element form at least a portion of a single tubular structure.
33. The stent delivery system of any one of Embodiments 25 to 28 or 30-32, wherein a proximal portion of the distal stent-engaging element and a proximal portion of the proximal stent-engaging element comprise a same diameter.
34. The stent delivery system of any one of Embodiments 25 to 33, wherein the distal stent-engaging element and the proximal stent-engaging element comprise a same length.
35. The stent delivery system of any one of Embodiments 25 to 34, wherein a proximal portion of the distal stent-engaging element comprises longitudinally extending struts, and wherein a proximal portion of the proximal stent-engaging element comprises longitudinally extending struts.
36. The stent delivery system of Embodiment 35, wherein a proximal portion of the distal stent-engaging element is circumferentially displaced from the proximal stent-engaging element such that each of the struts of the distal stent-engaging element is positioned between the struts of the proximal stent-engaging element.
37. The stent delivery system of any one of Embodiments 25 to 36, wherein the first radially outwardly projecting portion has a same width as the second radially outwardly projecting portion.
38. The stent delivery system of any one of Embodiments 25 to 37, wherein each of the first radially outwardly projecting portion and the second radially outwardly projecting portion comprises at least one prong.
39. The stent delivery system of Embodiment 38, wherein the at least one prong comprises a plurality of prongs.
40. The stent delivery system of any one of Embodiments 25 to 39, wherein the first radially outwardly projecting portion is circumferentially displaced from the second radially outwardly projecting portion.
41. The stent delivery system of any one of Embodiments 25 to 40, wherein the first radially outwardly projecting portion and the second radially outwardly projecting portion are configured to be radially compressed by the elongate outer member.
42. The stent delivery system of any one of Embodiments 25 to 41, wherein the pusher assembly comprises a guidewire channel.
43. The stent delivery system of any one of Embodiments 25 to 41, further comprising a guidewire channel positioned radially inward of the pusher assembly, the guidewire channel comprising a guidewire lumen.
44. The stent delivery system of any one of Embodiments 25 to 43, wherein longitudinal movement of the distal stent-engaging element and the proximal stent-engaging element is driven by a hydraulic assembly.
45. The stent delivery system of any one of Embodiments 25 to 44, further comprising a user-actuatable element configured to distally drive the distal stent-engaging element and the proximal stent-engaging element.
46. A stent delivery system comprising:
   an elongate outer member defining an outer member lumen; and
   a pusher assembly comprising:
      a plurality of stent-engaging elements arranged along a longitudinal axis and configured to telescopically move relative to each other, each of the plurality of stent-engaging elements comprising a radially outwardly projecting portion configured to engage a stent.
47. The stent delivery system of Embodiment 46, wherein each of the radially outwardly projecting portions comprises at least one prong.
48. The stent delivery system of Embodiment 46 or 47, wherein at least one of the radially outwardly projecting portions comprises a plurality of prongs.
49. The stent delivery system of any one of Embodiments 46 to 48, wherein the radially outwardly projecting portions are circumferentially displaced from each other.
50. The stent delivery system of any one of Embodiments 46 to 49, wherein the plurality of stent-engaging elements are configured to move simultaneously.
51. The stent delivery system of any one of Embodiments 46 to 50, wherein a subset of the plurality of stent-engaging elements are configured to move simultaneously, while the remainder of the plurality of stent-engaging elements are not moved.
52. The stent delivery system of any one of Embodiments 46 to 51, wherein a proximal portion of each of the stent-engaging elements is tubular.
53. The stent delivery system of any one of Embodiments 46 to 52, wherein each of the stent-engaging elements comprises a same length.
54. The stent delivery system of any one of Embodiments 46 to 52, wherein proximal portions of each of the stent-engaging elements form a single tubular structure.
55. The stent delivery system of any one of Embodiments 46 to 54, wherein a proximal portion of each of the stent engaging elements comprises a same diameter.
56. The stent delivery system of any one of Embodiments 46 to 55, wherein each of the stent-engaging elements comprises longitudinally extending struts.
57. The stent delivery system of Embodiment 56, wherein longitudinal struts of each of the stent-engaging elements are circumferentially displaced from each other.
58. The stent delivery system of any one of Embodiments 46 to 57, wherein the radially outwardly projecting portions comprise a same width.
59. The stent delivery system of any one of Embodiments 46 to 58, wherein the radially outwardly projecting portions are configured to be radially compressed by the elongate outer member.
60. The stent delivery system of any one of Embodiments 46 to 59, wherein the pusher assembly comprises a guidewire channel.
61. The stent delivery system of any one of Embodiments 46 to 59, further comprising a guidewire channel positioned radially inward of the pusher assembly, the guidewire channel comprising a guidewire lumen.
62. The stent delivery system of any one of Embodiments 46 to 61, wherein longitudinal movement of the plurality of stent-engaging elements is driven by a hydraulic assembly.
63. The stent delivery system of any one of Embodiments 46 to 62, further comprising a user-actuatable element configured to distally drive the plurality of stent-engaging elements.
64. A method of delivering a self-expanding stent at a treatment site using a stent delivery system, the method comprising:
   advancing the stent delivery system proximate to the treatment site, the stent delivery system comprising:
      an elongate outer member defining an outer member lumen;
      a pusher assembly in the outer member lumen, the pusher assembly comprising:
         a first stent-engaging element; and
         a second stent-engaging element, a distal end of the first stent-engaging element positioned distal to the second stent-engaging element, the first stent-engaging element configured to move independently from the second stent-engaging element; and
      the self-expanding stent radially between the elongate outer member and the pusher assembly in a radially compressed configuration;
   advancing a first portion of the self-expanding stent out of the elongate outer member by distally driving the first stent-engaging element out of the elongate outer member; and
   after advancing the first portion of the self-expanding stent, advancing a second portion of the self-expanding stent out of the elongate outer member by distally driving the second stent-engaging element out of the elongate outer member, while not distally driving the first stent-engaging element.
65. The method of Embodiment 64, wherein advancing the first portion of the self-expanding stent comprises distally driving the first stent-engaging element without driving the first stent-engaging element out the elongate outer member.
66. The method of Embodiment 64 or 65, further comprising advancing a third portion of the self-expanding stent out of the elongate outer member by driving a third stent-engaging element out of the outer member.
67. The method of Embodiment 66, wherein advancing the third portion of the self-expanding stent comprises, after advancing the second portion of the self-expanding stent, distally driving the third stent-engaging element, while not distally driving the second stent-engaging element.
68. The method of Embodiment 66 or 67, wherein advancing the third portion of the self-expanding stent comprises simultaneously distally driving the second stent-engaging element and the third stent-engaging element.
69. The method of any one of Embodiments 66 to 68, wherein the third portion of the self-expanding stent comprises a proximal end of the self-expanding stent.
70. The method of any one of Embodiments 64 to 68, wherein the self-expanding stent is woven to form wire intersections, and wherein engaging the self-expanding stent comprises engaging wire intersections of the stent with the first stent-engaging element.
71. The method of any one of Embodiments 64 to 70, wherein advancing the second portion of the stent comprises advancing the second portion of the stent over the first stent-engaging element.

What is claimed is:

1. A method of delivering a self-expanding stent at a treatment site using a stent delivery system, the method comprising:
advancing the stent delivery system proximate to the treatment site, the stent delivery system comprising:
an elongate outer member defining an outer member lumen;
a pusher assembly in the outer member lumen, the pusher assembly comprising a plurality of stent-engaging elements, the plurality of stent-engaging elements comprising a distal stent-engaging element, at least one intermediate stent-engaging element, and a proximal stent-engaging element, the plurality of stent-engaging elements being positioned coaxially relative to each other, each of the plurality of stent-engaging elements being configured to move independently relative to the other stent-engaging elements; and
the self-expanding stent radially between the elongate outer member and the pusher assembly in a radially compressed configuration;
advancing the plurality of stent-engaging elements until the plurality of stent engaging elements are positioned at a distal portion of the outer lumen, wherein advancing the plurality of stent-engaging elements comprises:
distally driving each of the at least one intermediate stent-engaging elements and the proximal stent-engaging element, while not distally driving any of the plurality of stent-engaging elements distal to the stent-engaging element being driven.

2. The method of claim 1, wherein advancing the plurality of stent-engaging elements comprises simultaneously distally driving the plurality of stent-engaging elements.

3. The method of claim 1, wherein advancing the plurality of stent-engaging elements comprises distally driving the plurality of stent-engaging elements toward a distal portion of the outer lumen without driving the plurality of stent-engaging elements out the elongate outer member.

4. The method of claim 3, further comprising, after distally driving the plurality of stent-engaging elements toward the distal portion of the outer lumen, simultaneously driving the plurality of stent-engaging elements out of the elongate outer member.

5. The method of claim 1, wherein advancing the plurality of stent-engaging elements comprises engaging the self-expanding stent with each of the stent-engaging elements.

6. The method of claim 5, wherein the self-expanding stent is woven to form wire intersections, and wherein engaging the self-expanding stent comprises engaging wire intersections of the stent with each of the stent-engaging element.

7. The method of claim 1, wherein distally driving each of the at least one intermediate stent-engaging elements and the proximal stent-engaging elements comprises driving a portion of the self-expanding stent over the distal stent-engaging element.

8. The method of claim 1 further comprising retracting the self-expanding stent.

9. The method of claim 8, wherein retracting the self-expanding stent comprises retracting the plurality of stent-engaging elements.

10. The method of claim 1, wherein advancing the plurality of stent-engaging elements comprises hydraulically driving the plurality of stent-engaging elements.

11. The method of claim 1, wherein advancing the plurality of stent-engaging elements comprises actuating a user-actuatable element configured to distally drive the plurality of stent-engaging elements.

12. A stent delivery system comprising:
an elongate outer member defining an outer member lumen;
a pusher assembly in the outer member lumen, the pusher assembly comprising:
a distal stent-engaging element comprising a first radially outwardly projecting portion configured to engage a first portion of a self-expanding stent; and
a proximal stent-engaging element comprising a second radially outwardly projecting portion configured to engage a second portion of the self-expanding stent, the second portion being proximal to the first portion of the stent-engaging element,
wherein the distal stent-engaging element is configured to move independently from the proximal stent-engaging element.

13. A method of delivering a self-expanding stent at a treatment site using a stent delivery system, the method comprising:
advancing the stent delivery system proximate to the treatment site, the stent delivery system comprising:
an elongate outer member defining an outer member lumen;
a pusher assembly in the outer member lumen, the pusher assembly comprising:
a first stent-engaging element; and
a second stent-engaging element, a distal end of the first stent-engaging element positioned distal to the second stent-engaging element, the first stent-engaging element configured to move independently from the second stent-engaging element; and
the self-expanding stent radially between the elongate outer member and the pusher assembly in a radially compressed configuration;
advancing a first portion of the self-expanding stent out of the elongate outer member by distally driving the first stent-engaging element out of the elongate outer member; and
after advancing the first portion of the self-expanding stent, advancing a second portion of the self-expanding stent out of the elongate outer member by distally driving the second stent-engaging element out of the elongate outer member, while not distally driving the first stent-engaging element.

14. The method of claim 13, wherein advancing the first portion of the self-expanding stent comprises distally driving the first stent-engaging element without driving the first stent-engaging element out the elongate outer member.

15. The method of claim 13, further comprising advancing a third portion of the self-expanding stent out of the elongate outer member by driving a third stent-engaging element out of the outer member.

16. The method of claim 15, wherein advancing the third portion of the self-expanding stent comprises, after advancing the second portion of the self-expanding stent, distally driving the third stent-engaging element, while not distally driving the second stent-engaging element.

17. The method of claim 15, wherein advancing the third portion of the self-expanding stent comprises simultaneously distally driving the second stent-engaging element and the third stent-engaging element.

18. The method of claim 15, wherein the third portion of the self-expanding stent comprises a proximal end of the self-expanding stent.

19. The method of claim 13, wherein the self-expanding stent is woven to form wire intersections, and wherein engaging the self-expanding stent comprises engaging wire intersections of the stent with the first stent-engaging element.

20. The method of claim 13, wherein advancing the second portion of the stent comprises advancing the second portion of the stent over the first stent-engaging element.
